(19) United States
(12) Patent Application Publication
(10) Pub. No.: US 2010/0305615 A1
(43) Pub. Date: Dec. 2, 2010

(54) Multi-level Polyaxial Screw Connection Mechanism

(75) Inventors: Mohamed Ahmed Hafez Ramadan, Cairo (EG); Mahmoud F. Abdelgany, Rockaway, NJ (US)

Correspondence Address: Rahman LLC;Mohammad Rahman, 10025 Governor Warfield Parkway, Suite 110, Columbia, MD, 21044, US

(73) Assignee: Custom Spine, Inc., Parsippany, NJ (US)

(21) Appl. No.: 12/474,970

(22) Filed: May 29, 2009

(51) Int. Cl. A61B 17/70; A61B 17/86

(52) U.S. Cl. 606/264; 606/305

(57) ABSTRACT

A multi-level polyaxial screw connection mechanism includes a bone fixation component and a head component directly connected to the bone fixation component. The head component includes a lower portion comprising at least one lower portion concave socket configured and an upper portion elevated compared with the lower portion. The upper portion comprises at least one upper portion concave socket configured and the lower portion(s) concave socket comprises a volume substantially equal to a volume of the upper portion(s) concave socket.

### BACKGROUND

1. Technical Field

The embodiments herein generally relate to medical devices and assemblies, and more particularly to an orthopedic surgical implant assembly used in the field of surgical lumbar, thoracic, and cervical spine treatment.

2. Description of the Related Art

Surgical procedures treating spinal injuries are one of the most complex and challenging surgeries for both the patient and the surgeon. When there are various deformities, trauma, or fractures of the vertebra, surgeons may attempt to “fuse” them together by attaching screw-like devices into the pedicles of the spine and thereby connecting several vertebrae (typically two or more) using a semi-rigid rod. Due to the complexity of the human anatomy, however, the vertebra to be fused may not reside at the same elevation, or height, along a longitudinal axis within the human body. To reach multiple heights during the fusing procedure, most surgeons bend the rod or more rods (causing notches thereby reducing fatigue resistance) before placing them into two or more non-aligned pedicle screws in order to properly stabilize the pedicle screw assembly within the patient's body.

Depending on the purpose of the spine surgery, indications, and patient size, surgeons may pre-operatively choose between different spinal systems with differing rod sizes sometimes causing delays in surgery while waiting for more adequate systems to be sterilized. Some surgeons prefer monoaxial screws for rigidity, while some sacrifice rigidity for surgical flexibility in screw placement.

Additionally, conventional systems require several different components to be manipulated and assembled by the surgeon during a surgical procedure. Reducing the number of components in a screw assembly that are manipulated and assembled would simplify operating room logistics; the steps performed by the surgeon during the surgical procedure; and, ultimately, improve patient recovery time.

### SUMMARY

In view of the foregoing, an embodiment herein provides a multi-level bone fixation device comprising a bone fixation component; and a head component directly connected to the bone fixation component, wherein the head component comprises a lower portion comprising at least one lower portion concave socket configured therein; and an upper portion elevated compared with the lower portion, wherein the upper portion comprises at least one upper portion concave socket configured therein, wherein the at least one lower portion concave socket comprises a volume substantially equal to a volume of the at least one upper portion concave socket.

Such an embodiment further provides that the bone fixation component may comprise any of a bone anchor and a bone screw. In addition, at least one lower portion concave socket may comprise two lower portion concave sockets configured at the same level.

An assembly is also provided that comprises a first bone fixation device comprising a first bone fixation component; and a first head component directly connected to the first bone fixation component, wherein the first head component comprises a lower portion comprising a plurality of lower portion concave sockets configured therein; and an upper portion elevated compared with the lower portion, wherein the upper portion comprises at least one upper portion concave socket configured therein; at least one longitudinal member comprising at least one fastener channel bored therethrough and at least one outwardly protruding and expandable round bulbous body that connects to at least one of (i) the plurality of lower portion concave sockets and (ii) the at least one upper portion concave socket; and a second bone fixation device operatively connected to the at least one longitudinal member, wherein the second bone fixation device comprises a second bone fixation component; and a second head component directly connected to the second bone fixation component, wherein the second head component comprises at least one concave socket that receives a bulbous body.

In such an assembly, the second head component of the second bone fixation device may also comprise a first level portion comprising a plurality of first level portion concave sockets configured therein; and a second level portion comprising an elevated surface compared with the first level portion, wherein the second level portion comprises at least one second level portion concave socket configured therein. In addition, the second head component of the second bone fixation device may comprise exactly one concave socket. Furthermore, such an assembly may further comprise at least one pin, wherein each pin engages within one fastener channel and contacts one bulbous body causing the one bulbous body to outwardly expand. Additionally, any of the first bone fixation device and the second bone fixation device in such an assembly may comprise any of a bone anchor and a bone screw. At least one lower portion concave socket of such an assembly may comprise two lower portion concave sockets configured at the same level. Moreover, at least one longitudinal member of such an assembly may comprise a multi-component member. Furthermore, at least one longitudinal member of such an assembly may comprise a telescoping member.

An apparatus is also provide that comprises a pair of multi-level bone fixation devices each comprising a first bone fixation component; and a first head component directly connected to the first bone fixation component, wherein the first head component comprises a lower portion comprising a pair of lower portion concave sockets configured therein; and an upper portion elevated compared with the lower portion, wherein the upper portion comprises an upper portion concave socket configured therein; a pair of co-linear longitudinal members each comprising a first pair of fastener channels bored therethrough; and a first pair of outwardly protruding and expandable round bulbous bodies, wherein a first round bulbous body of each co-linear longitudinal member connects to one of the lower portion concave sockets; a pair of single-level bone fixation devices each comprising a second bone fixation component; and a second head component directly connected to the second bone fixation component, wherein the second head component comprises a concave socket configured therein; a transverse longitudinal member that is positioned transverse to the pair of co-linear longitudinal members, wherein the transverse longitudinal member comprises a second pair of fastener channels bored therethrough; and a second pair of outwardly protruding and expandable round bulbous bodies, wherein a first round bulbous body of the transverse longitudinal member connects to the upper portion concave socket; a plurality of pins engaging the first and second pair of fastener channels.

In such an apparatus, each pin may engage a bulbous body causing the bulbous body to outwardly expand. Furthermore, any of the pair of multi-level bone fixation devices and the pair of single-level bone fixation devices in such an apparatus may comprise any of a bone anchor and a bone screw. Additionally, the pair of lower portion concave sockets may be configured at the same level in such an apparatus. The concave socket of each single-level bone fixation device of such an apparatus may be at the same level as the level of the pair of lower portion concave sockets. In addition, any of (i) the pair of co-linear longitudinal members and (ii) the transverse longitudinal member may comprise a multi-component member. Any of (i) the pair of co-linear longitudinal members and (ii) the transverse longitudinal member may also comprise a telescoping member. In such an apparatus, the first head component may be larger than the second head component. Moreover, all sockets may comprise identical volumes.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a perspective view of a multi-level screw connection assembly according to an embodiment described herein;

FIG. 2 illustrates a perspective view of a multi-level fixation component according to an embodiment described herein;

FIG. 3(A) illustrates a perspective view of a longitudinal member according to a first embodiment described herein;

FIG. 3(B) illustrates a perspective view of a longitudinal member according to a second embodiment described herein;

FIG. 3(C) illustrates a perspective view of another longitudinal member according to a third embodiment described herein;

FIG. 4 illustrates a cross-sectional view of a longitudinal member according to an embodiment described herein;

FIG. 5 illustrates a bottom view of the bulbous end of a longitudinal member according to an embodiment described herein;

FIG. 6(A) illustrates a perspective view of a securing pin according to an embodiment described herein;

FIG. 6(B) illustrates a perspective view of a set screw according to an embodiment described herein;

FIG. 6(C) illustrates a top view of a set screw according to an embodiment described herein; and

FIG. 7 illustrates a perspective view of two adjoining longitudinal members according to an embodiment described herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

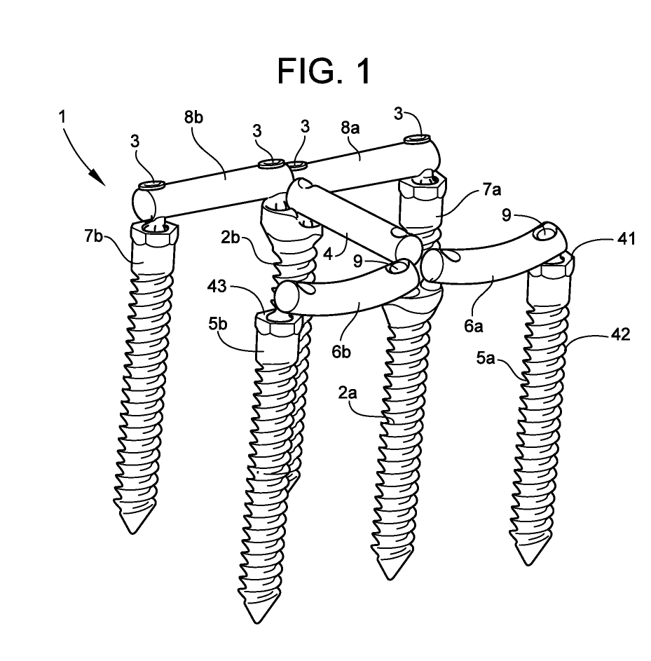

The embodiments herein provide an improved multi-level headless polyaxial screw device with fewer components than conventional systems. Referring now to the drawings and, more particularly to FIGS. 1 through 7, where similar reference characters denote corresponding features consistently throughout the figures, there are shown preferred embodiments.

FIG. 1 illustrates an embodiment of a multi-level polyaxial screw connection assembly 1. In one embodiment, multi-level connection system 1 includes multi-level fixation components 2a, 2b, a plurality of securing pins 3, single-level fixation components 5a, 5b, 7a, 7b, longitudinal members 4, 6a, 6b, 8a, 8b and a plurality of securing channels 9. As shown in FIG. 1, multi-level fixation components 2a, 2b are joined by longitudinal member 4. Multi-level fixation component 2a is attached and secured to bone (not shown) at a first height (or level), while multi-level fixation component 2b is attached and secured to bone (not shown) at a second height (or level). Consequently, multi-level fixation components 2a, 2b are secured to bone at different heights, with respect to one another, to accommodate variation in human anatomy. Additionally, multi-level fixation component 2a is adjacent to fixation components 5a, 5b and connected thereto by longitudinal members 6a, 6b, respectively, via securing pins 3 mounted into securing channels 9 (described in further detail below). Multi-level fixation component 2b is adjacent to fixation components 7a, 7b and connected thereto by longitudinal members 8a, 8b via securing pins 3 mounted into securing channels 9 (described in further detail below). In the configuration shown in FIG. 1, fixation components 5a, 5b are secured to bone (not shown) at the same height as multi-level fixation component 2a and fixation components 7a, 7b are secured to bone (not shown) at the same height at multi-level fixation component 2b. The configuration of the different components shown in FIG. 1, however, is one of many possible embodiments of the herein. Based on what is shown in FIG. 1 and discussed below, those skilled in the art could readily implement alternative configurations without undue experimentation and the embodiments herein are not limited to the configuration shown in FIG. 1.

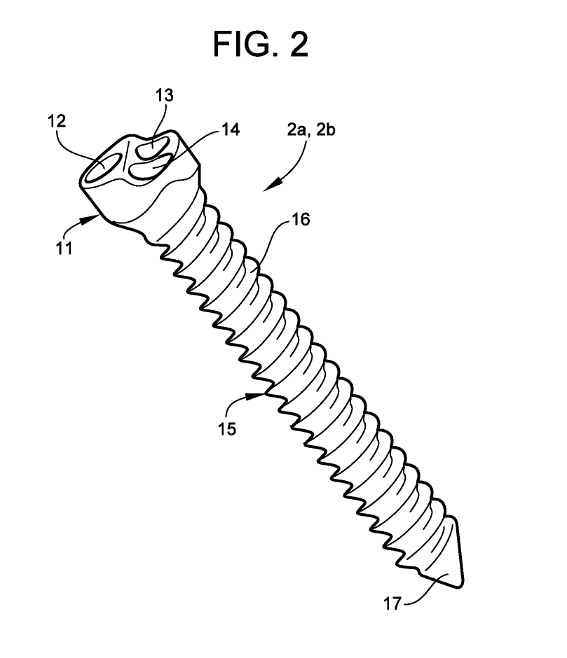

FIG. 2, with reference to FIG. 1, is a perspective view of a multi-level fixation component 2a, 2b. In the embodiment shown, multi-level fixation component 2a, 2b includes upper portion 11, first socket 12, second socket 13, lateral socket 14, lower portion 15, threads 16, and tip 17. FIG. 2 shows upper portion 11 of multi-level fixation component 2a, 2b having three female spherical sockets: first socket 12, second socket 13, and lateral socket 14. Each socket 12, 13, 14 is configured to allow a longitudinal member 4, 6a, 6b, 8a, 8b to pivot freely but not to disassemble once a bulbous body (described below) is inserted and engaged within the respective socket 12, 13, 14. In one embodiment of multi-level fixation component 2a, 2b, the maximum angulation for each socket is 25 degrees/side, but the medial correction/travel of a longitudinal member (not shown) is 3.2 mm/side, which is nearly 50% more than what most conventional screws offer.

Situated below upper portion 11, lower portion 15 includes threads 16 to engage different biological matter. While lower portion 15 is illustrated as a bone screw, those skilled in the art would understand that other types of fixation components could be utilized (such as a bone anchor) in accordance with the embodiments herein. The threads 16 of multi-level fixation component 2a, 2b may be a multiple lead thread to allow faster insertion into a bone. Threads 16 may also be tapered on the minor diameter while cylindrical on the major diameter to allow a new “bite” with every turn and to accommodate more thread depth towards the bottom of multi-level fixation component 2a, 2b for the cancellous bone. For example, threads 16 may be double lead, which provides greater surface contact with the bone, but drives at 4 mm/revolution. Lower portion 15 also includes a tapered tip 17 to assist engagement of multi-level fixation component 2a, 2b into different biological matter.

Additionally, as indicated in FIG. 1, each single-level fixation component 5a, 5b, 7a, 7b also includes an upper portion 41 and a lower portion 42. However, unlike multi-level fixation component 2a, 2b, the single-level fixation components comprise only a single socket 43 in the upper portion 41 thereof. The features described above for lower portion 15 of multi-level fixation component 2a, 2b, however, are similar for the lower portion of the single-level fixation components 5a, 5b, 7a, 7b.

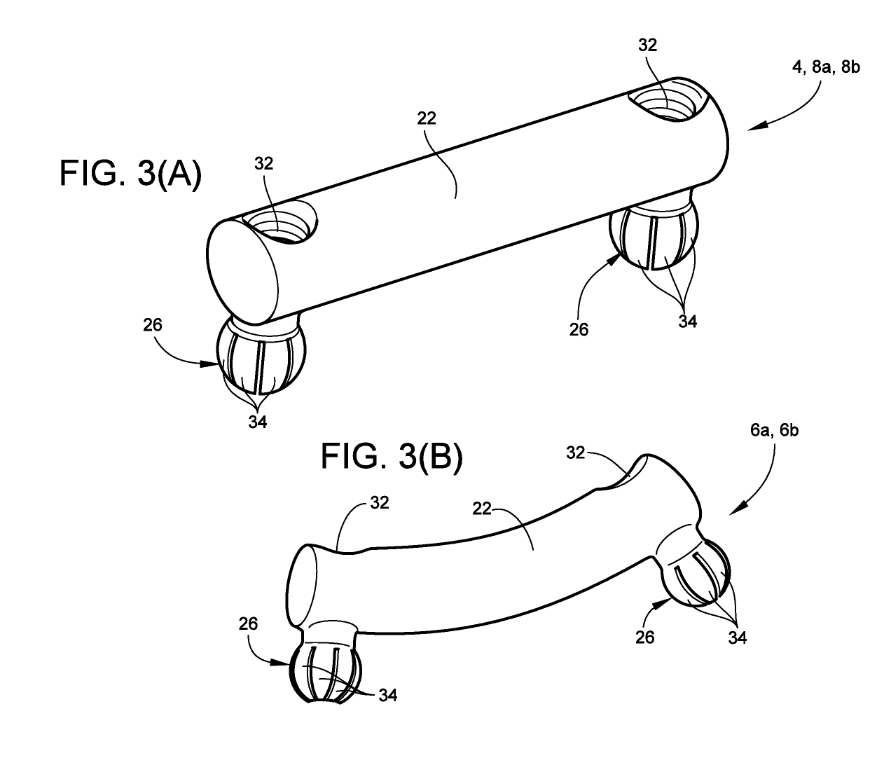

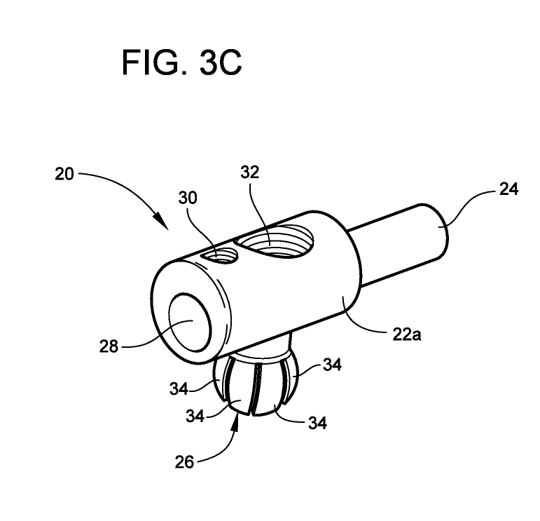

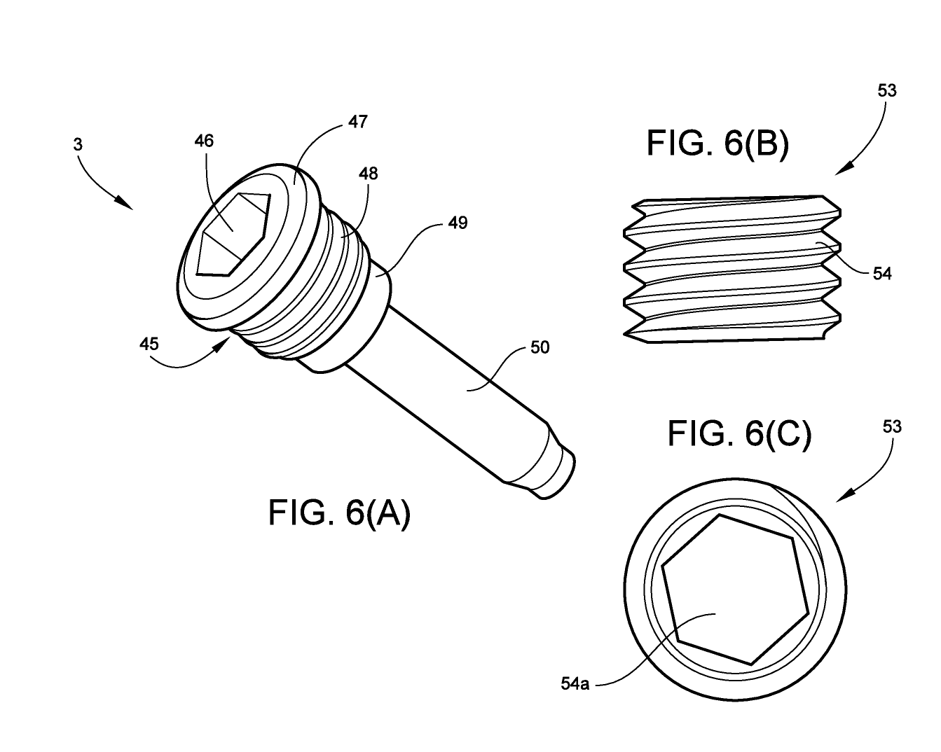

FIG. 3(A), with reference to FIGS. 1 and 2, provides a front perspective view of an embodiment of longitudinal members 4, 8a, 8b. FIG. 3(B), with reference to FIGS. 1 and 2, provides a front perspective view of an embodiment of longitudinal members 6a, 6b. Longitudinal members 4, 6a, 6b, 8a, 8b each include a first body 22 and a bulbous body 26 directly connected to the first body 22. Bored into first body 22 is securing channel 32, shown with threading etched into its inner perimeter. Furthermore, bulbous body 26 includes a plurality of slotted flanges 34 that allow bulbous body 26 to expand when engaged within a female socket 12, 13, 14, 43 of either a single-level fixation component 5a, 5b, 7a, 7b or a multi-level fixation component 2a, 2b, respectively, at any allowable angle once securing pin 3 (shown in FIG. 6(A)) is urged through the securing channel 32 and causing the bulbous body 26 to expand. Since the bulbous body 26 is pivoting inside a female socket 12, 13, 14, 43, connection assembly 1 is allowed to be inserted deeper into the bone without having the bone or anatomy prematurely limit the range of angulations of longitudinal member 20.

FIG. 3(C), with reference to FIGS. 1 and 2, provides a front perspective view of another embodiment of a longitudinal member 20, which may be used to connect to the fixation components 2a, 2b, 5a, 5b, 7a, 7b. In FIG. 3(C), longitudinal member 20 includes a first body 22a, an elongated body 24, and a bulbous body 26 that is directly connected to the first body 22a. Bored in first body 22a are a plurality of channels including elongation channel 28, optional setting channel 30, and securing channel 32. As shown, elongation channel 28 has a smooth bore while both setting channel 30 and securing channel 32 each have threads etched into an inner perimeter therein. Additionally, bulbous body 26 includes a plurality of slotted flanges 34 that allow bulbous body 26 to expand when engaged within a female socket 12, 13, 14, 43 of either a single-level fixation component 5a, 5b, 7a, 7b or a multi-level fixation component 2a, 2b, respectively, at any allowable angle once securing pin 3 (shown in FIG. 6(A)) is urged through the securing channel 32 and causing the bulbous body 26 to expand. Since longitudinal member 20 is pivoting inside a female socket 12, 13, 14, 43, connection assembly 1 is allowed to be inserted deeper into the bone without having the bone or anatomy prematurely limit the range of angulations of longitudinal member 20.

Elongation channel 28 is preferably configured as a substantially horizontal bore (i.e., with respect to the longitudinal axis of the first body 22a and elongated body 24) through the first body 22a and terminates at the securing channel 32. Setting channel 30 is a substantially vertical bore (i.e., with respect to the longitudinal axis of the first body 22a and elongated body 24) through the first body 22a and terminates at elongation channel 28. Furthermore, securing channel 32 is also a substantially vertical bore (i.e., with respect to the longitudinal axis of the first body 22a and elongated body 24), and is configured through first body 22a and bulbous body 26.

Longitudinal member 20 also has threads 35 etched into first body 22a, configured to mate with threads embedded in securing pin 3 (as described below). Setting channel 30 is similarly configured with threads etched into first body 22 and are configured to mate with threads embedded on a set screw 53 (shown in FIG. 6(B)). Longitudinal member 20 may connect with another similarly configured longitudinal member 20 such that the elongated body 24 of the second longitudinal member is seated within elongation channel 28 of the first longitudinal member with a set screw 53 being inserted in setting channel 30 to lock the two longitudinal members together once the proper alignment is selected by the surgeon.

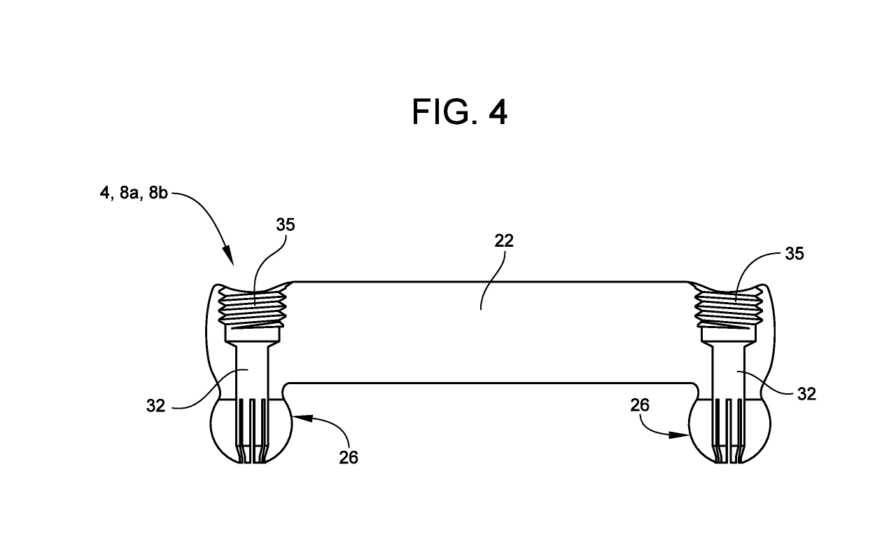

FIG. 4, with reference to FIGS. 1 through 3(C), illustrates a cross-sectional view of longitudinal members 4, 8a, 8b. Longitudinal members 6a, 6b, while not shown in FIG. 4, have a similar cross-section. FIG. 4 shows securing channel 32 is bored through both first body 22 and bulbous body 26. In addition, FIG. 4 shows securing channel 32 is etched with threads 35. While not shown, threads 35 are configured to mate with securing pin 3 (shown in FIG. 6(A)).

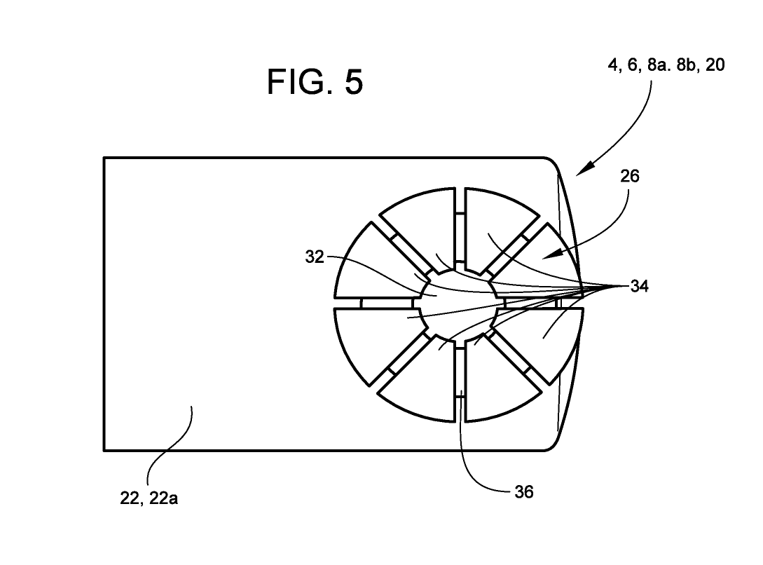

FIG. 5, with reference to FIGS. 1 through 4, illustrates the bottom view of any of the longitudinal members 4, 6a, 6b, 8a, 8b, 20, and is shown to include bulbous body 26 directly connected to first body 22, 22a. As shown in FIG. 5, longitudinal members 4, 6a, 6b, 8a, 8b, 20 include the expandable bulbous (or generally spherical) male body 26 for engaging a concave female socket 12, 13, 14, 43 of either a single-level fixation component 5a, 5b, 7a, 7b or a multi-level fixation component 2a, 2b, respectively. A plurality of axially spaced slots 36 are cut into bulbous body 26 forming a plurality of flanges 34, which expand once securing pin 3 (shown in FIG. 6(A)) is forced through securing channel 32 of first body 22, 22a and cause the flanges 34 to outwardly project and expand. As a consequence, bulbous body 26 expands into the female socket 12, 13, 14, 43 of either a single-level fixation component 5a, 5b, 7a, 7b or a multi-level fixation component 2a, 2b, respectively, at any allowable angle and thereby securing longitudinal members 4, 6a, 6b, 8a, 8b, 20 to either a single-level fixation component 5a, 5b, 7a, 7b or a multi-level fixation component 2a, 2b.

FIG. 6(A), with reference to FIGS. 1 through 5, illustrates a perspective view of securing pin 3. As shown, securing pin 3 includes an upper fastening portion 45 and a lower tip portion 50. Upper fastening portion 45 further includes fastening socket 46, pin head 47, threads 48, and connecting ring 49. As shown, fastening socket 46 is a hexagonal shape. Those skilled in the art would recognize that other configurations are possible—for example, fastening socket 46 may be square or any other polygonal shape or may be a linear slit or cross-slit in pin head 47. Threading 48 is embedded around an outer perimeter of upper fastening portion 45 and is configured to engage threads 35 etched into the inner perimeter of securing channel 32 of the longitudinal members 4, 6a, 6b, 8a, 8b, 20. Connecting ring 49 is coupled to both the upper fastening portion 45 and lower tip portion 50. When upper fastening portion 45 and lower tip portion 50 are composed of different materials (as described in further detail below), connecting ring 49 provides additional strength in the coupling thereof.

Securing pin 3 may also comprise a multi-part assembly. For example, the upper fastening portion 45 of securing pin 3 may comprise titanium and the lower tip portion 50 of the securing pin 3 may comprise a ceramic material. Additionally, the lower tip portion 50 may comprise a mechanically harder material than the upper fastening portion 45. In such a configuration, longitudinal members 4, 6a, 6b, 8a, 8b, 20 and either a single-level fixation component 5a, 5b, 7a, 7b or a multi-level fixation component 2a, 2b may optionally comprise a first material, and the lower tip portion 50 of the pin 3 may comprise a material having a higher material hardness and compressive yield strength than the first material. Moreover, connection assembly 1 may further comprise a wear resistant ceramic coating (not shown) over longitudinal members 4, 6a, 6b, 8a, 8b, 20 and either a single-level fixation component 5a, 5b, 7a, 7b or a multi-level fixation component 2a, 2b.

Connection assembly 1 can also be used as a dynamic rod system to complement artificial discs. According to this aspect of the embodiments herein, the outside of the bulbous body 26 and an inner spherical surface of female sockets 12, 13, 14, 43 are coated with a wear resistant ceramic coating. In this scenario, the securing pin 3 is not digging into the socket and in fact is configured at a shorter length than some of the other embodiments. This allows some motion instead of rigid fixation and shares the load with the artificial disc disallowing excessive forces being applied to the artificial disc and increasing its functional life. For example, this occurs as a result of the ceramic coating, which may be used in the embodiments herein. As such, the bulbous body 26 of longitudinal members 4, 6a, 6b, 8a, 8b, 20 and the female sockets 12, 13, 14, 43 has a lower friction and higher wear resistance characteristics, thus improving the overall movement characteristics of connection assembly 1.

FIG. 6(B), with reference to FIGS. 1 through 6(A), illustrates a set screw 53 used in accordance with the embodiments herein. The set screw 53 is dimensioned and configured to engage the setting channel 30 of the longitudinal member 20. The set screw 53 is used to secure a corresponding elongated body 24 of another longitudinal member 20, which connects inside elongated channel 28 to link two longitudinal members 20 together. Once the elongated body 24 of another longitudinal member 20 is positioned inside the elongation channel 28 and is in a desired position, the set screw 53 is inserted into the setting channel 30 such that the set screw 53 engages the elongated body 24 of another longitudinal member 20 and fixes it into a static position. Accordingly, set screw 53 is embedded with thread 54, which are configured to mate with threads etched in setting channel 30 (shown in FIG. 3(C)). In addition, set screw 53 also includes socket 54a to assist in engaging the set screw 53 with the setting channel 30. As shown, socket 54a is a hexagonal shape. Those skilled in the art would recognize that other configurations are possible—for example, socket 54a may be square or any other polygonal shape or may be a linear slit or cross-slit across set screw 53.

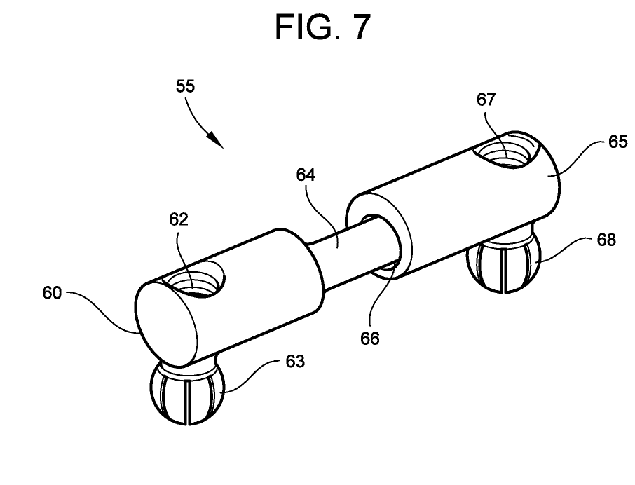

FIG. 7, with reference to FIGS. 1 through 6(B) illustrates a second embodiment of a longitudinal member system 55. Longitudinal member system 55 includes two adjoining longitudinal members 60, 65 connected together. The elongation system includes male longitudinal member 60 and female longitudinal member 65. As shown, male longitudinal member 60 includes securing channel 62 positioned through the member 60 and through connected bulbous body 63, which then connects to either a single-level fixation component 5a, 5b, 7a, 7b or a multi-level fixation component 2a, 2b via a securing pin 3. Female longitudinal member 65 includes an elongation channel 66 and securing channel 67 positioned through the member 65 and through connected bulbous body 68, which then connects to either a single-level fixation component 5a, 5b, 7a, 7b or a multi-level fixation component 2a, 2b via a securing pin 3. Also shown in FIG. 7 is elongated member 64 of male longitudinal member 60 engaged with the elongation channel 66 of female longitudinal member 65. While not shown in FIG. 7, a corresponding setting channel and set screw may be incorporated with member 65 in a manner consistent with the embodiments shown in FIGS. 1 through 6(C).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A multi-level bone fixation device comprising:
a bone fixation component; and
a head component directly connected to said bone fixation component, wherein said head component comprises:
a lower portion comprising at least one lower portion concave socket configured therein; and
an upper portion elevated compared with said lower portion, wherein said upper portion comprises at least one upper portion concave socket configured therein,
wherein said at least one lower portion concave socket comprises a volume substantially equal to a volume of said at least one upper portion concave socket.

2. The device of claim 1, wherein said bone fixation component comprises any of a bone anchor and a bone screw.

3. The device of claim 1, wherein said at least one lower portion concave socket comprises two lower portion concave sockets configured at the same level.

4. An assembly comprising:
a first bone fixation device comprising:
a first bone fixation component; and
a first head component directly connected to said first bone fixation component, wherein said first head component comprises:
a lower portion comprising a plurality of lower portion concave sockets configured therein; and
an upper portion elevated compared with said lower portion, wherein said upper portion comprises at least one upper portion concave socket configured therein;
at least one longitudinal member comprising at least one fastener channel bored therethrough and at least one outwardly protruding and expandable round bulbous body that connects to at least one of (i) said plurality of lower portion concave sockets and (ii) said at least one upper portion concave socket; and
a second bone fixation device operatively connected to said at least one longitudinal member, wherein said second bone fixation device comprises
a second bone fixation component; and
a second head component directly connected to said second bone fixation component, wherein said second head component comprises:
at least one concave socket that receives a bulbous body.

5. The assembly of claim 4, wherein said second head component of said second bone fixation device comprises:
a first level portion comprising a plurality of first level portion concave sockets configured therein; and
a second level portion comprising an elevated surface compared with said first level portion, wherein said second level portion comprises at least one second level portion concave socket configured therein.

6. The assembly of claim 4, wherein said second head component of said second bone fixation device comprises exactly one concave socket.

7. The assembly of claim 4, further comprising at least one pin, wherein each pin engages within one fastener channel and contacts one bulbous body causing said one bulbous body to outwardly expand.

8. The assembly of claim 4, wherein any of said first bone fixation device and said second bone fixation device comprises any of a bone anchor and a bone screw.

9. The assembly of claim 4, wherein said at least one lower portion concave socket comprises two lower portion concave sockets configured at the same level.

10. The assembly of claim 4, wherein said at least one longitudinal member comprises a multi-component member.

11. The assembly of claim 4, wherein said at least one longitudinal member comprises a telescoping member.

12. An apparatus comprising:
a pair of multi-level bone fixation devices each comprising:
a first bone fixation component; and
a first head component directly connected to said first bone fixation component, wherein said first head component comprises:
a lower portion comprising a pair of lower portion concave sockets configured therein; and
an upper portion elevated compared with said lower portion, wherein said upper portion comprises an upper portion concave socket configured therein;
a pair of co-linear longitudinal members each comprising:
a first pair of fastener channels bored therethrough; and
a first pair of outwardly protruding and expandable round bulbous bodies, wherein a first round bulbous body of each co-linear longitudinal member connects to one of the lower portion concave sockets;
a pair of single-level bone fixation devices each comprising:
a second bone fixation component; and
a second head component directly connected to said second bone fixation component, wherein said second head component comprises a concave socket configured therein;
a transverse longitudinal member that is positioned transverse to said pair of co-linear longitudinal members, wherein said transverse longitudinal member comprises:
a second pair of fastener channels bored therethrough; and
a second pair of outwardly protruding and expandable round bulbous bodies, wherein a first round bulbous body of said transverse longitudinal member connects to said upper portion concave socket;
a plurality of pins engaging the first and second pair of fastener channels.

13. The apparatus of claim 12, wherein each pin engages a bulbous body causing said bulbous body to outwardly expand.

14. The apparatus of claim 12, wherein any of said pair of multi-level bone fixation devices and said pair of single-level bone fixation devices comprise any of a bone anchor and a bone screw.

15. The apparatus of claim 12, wherein said pair of lower portion concave sockets are configured at the same level.

16. The apparatus of claim 15, wherein said concave socket of each single-level bone fixation device is at the same level as the level of said pair of lower portion concave sockets.

17. The apparatus of claim 12, wherein any of (i) said pair of co-linear longitudinal members and (ii) said transverse longitudinal member comprises a multi-component member.

18. The apparatus of claim 12, wherein any of (i) said pair of co-linear longitudinal members and (ii) said transverse longitudinal member comprises a telescoping member.

19. The apparatus of claim 12, wherein said first head component is larger than said second head component.

20. The apparatus of claim 12, wherein all sockets comprise identical volumes.
